(19) United States
(12) Patent Application Publication
(10) Pub. No.: US 2005/0154026 A1
(43) Pub. Date: Jul. 14, 2005

(54) Use of proton pump inhibitors for the treatment of noncardiac chest pain

(75) Inventor: Wolfgang-Alexander Simon, Konstanz (DE)

Correspondence Address: NATH & ASSOCIATES PLLC, 1030 FIFTEENTH STREET, N.W., SIXTH FLOOR, WASHINGTON, DC, 20005, US

(73) Assignee: Altana Pharma AG, Konstanz (DE)

(21) Appl. No.: 10/507,613

(22) PCT Filed: Mar. 11, 2003
(86) PCT No.: PCT/EP03/02466

(30) Foreign Application Priority Data
Mar. 15, 2002 (EP) 02005955.6

(51) Int. Cl. A61K 31/4439

(52) U.S. Cl. 514/338

(57) ABSTRACT

The invention relates to the use of proton pump inhibitors in the treatment of non-cardiac chest pain.

### TECHNICAL FIELD

The invention relates to medicaments comprising compounds from the class consisting of the acid secretion inhibitors which are to be used for the treatment of non-cardiac chest pain.

### PRIOR ART

A whole series of compounds are known from the prior art which inhibit gastric acid secretion by blocking the proton pump and which have therefore also been designated as proton pump inhibitors (PPI). These compounds are suitable for the treatment of gastric and intestinal disorders and, accordingly, some of them have been approved by health authorities.

### DESCRIPTION OF THE INVENTION

Surprisingly, it has now been found that the proton pump inhibitors, whose original field of use is the treatment of gastric and intestinal disorders, are particularly suitable for the treatment of non-cardiac chest pain.

The invention thus relates in a first aspect to the use of proton pump inhibitors in the treatment of non-cardiac chest pain.

Proton pump inhibitors are designated as those substances which inhibit gastric acid secretion by blocking the proton pump, i.e. substances which bind covalently to the H+/K+-ATPase, the enzyme responsible for gastric acid secretion. These include in particular active compounds having a 2-[(2-pyridinyl)methylsulphinyl]-1H-benzimidazole skeleton or related skeletons, where these skeletons may be substituted in various different ways. The term “proton pump inhibitor” according to the invention comprises not only the active compounds as such, but also their pharmacologically acceptable salts, solvates (in particular hydrates), etc.

Examples of proton pump inhibitors which may be mentioned are those described and claimed in the patent applications and patents below: DE-A-3531487, EP-A-0 005 129, EP-A-0 124 495, EP-A-0 166 287, EP-A0 174 726, EP-A-0 184 322, EP-A-0 254 588, EP-A-0 261 478, EP-A-1 268 956, EPA-0 434 999 and WO-A-9523149. Examples which may be mentioned here are the compounds 2-[2-(N-isobutyl-N-methylamino)benzylsulphinyl]benzimidazole (INN: leminoprazole), 2-(4-methoxy-6,7,8,9-tetrahydro-5H-cyclohepta[b]pyridin-9-ylsulphinyl)-1H-benzimidazole (INN: nepaprazole), 2-(4-methoxy-3-methylpyridin-2-ylmethylsulphinyl)5-pyrrol-1-yl-1H-benzimidazole (IY-81149), 5-methoxy-2-[(4-methoxy-3,5-dimethyl-2-pyridinyl)methylsulphinyl]-1H-imidazo[4,5-b]pyridine (tenatoprazole), especially 5-methoxy-2-[(4-methoxy-3,5-dimethyl-2-pyridinyl)methylsulphinyl]-1H-benzlmidazole (INN: omeprazole), 5-methoxy-2-[(S)-[(4-methoxy-3,5-dimethyl-2-pyridinyl)methyl]sulphinyl]-1H-benzmidazole (INN: esomeprazole), 2-[(3-methyl-4-(2,2,2-trifluoroethoxy)-2-pyridinyl)methylsulphinyl]-1H-benzimidazole (INN: lansoprazole) and 2-{[4-(3-methoxypropoxy)-3-methylpyridin-2-yl]-methyl-sulphinyl}-1H-benzimidazole (INN: rabeprazole) and in particular 5-difluoromethoxy-2-[(3,4-dimethoxy-2-pyridinyl)methylsulphinyl]-1H-benzimidazole (INN: pantoprazole) and (-)-5-difluoromethoxy-2-[(3,4-dimethoxy-2-pyridinyl)methylsulphinyl]-1H-benzimidazole [INN: (−)-pantoprazole].

The proton pump inhibitors are present as such or in the form of their salts with bases. Examples of salts with bases which may be mentioned are sodium, potassium, magnesium or calcium salts. If the proton pump Inhibitors or their salts are isolated in crystalline form, the crystals may contain variable amounts of solvent. Correspondingly, according to the invention, the term “proton pump inhibitor” also includes all solvates, in particular all hydrates, of the proton pump Inhibitors and their salts. Particularly preferred salts or hydrates of proton pump inhibitors which may be mentioned are pantoprazole-sodium sesquihydrate (=pantoprazole-sodium×1.5 H₂O), (−)-pantoprazole-sodium sesquihydrate, pantoprazole-magnesium dihydrate, omeprazole-magnesium, omeprazole-magnesium tetrahydrate, esomeprazole-magnesium and esomeprazole-magnesium tetrahydrate.

Non-cardiac chest pain to be treated which may be mentioned in particular is pain interpreted by the patient as a threatening acute or chronic disorder affecting the heart, which disorder is a consequence associated with anxiety and resulting secondary symptoms such as a feeling of tightness, fits of perspiration or tachykardia.

The invention relates in a further aspect to the use of proton pump inhibitors for the treatment of patients who are suffering from non-cardiac chest pain.

The invention further relates to a method for the treatment of non-cardiac chest pain which consists in administering to a patient who needs such a treatment an effective amount of a proton pump inhibitor.

The invention further relates to the use of proton pump inhibitors for the production of medicaments for the treatment of non-cardiac chest pain.

The invention further relates to a pharmaceutical preparation for the treatment of non-cardiac chest pain which contains a proton pump inhibitor as active compound. The invention further relates to a ready-to-use medicament, comprising a proton pump inhibitor as active compound, which contains a reference to the fact that this ready-to-use medicament can be employed for the treatment of non-cardiac chest pain.

Commercial Utility

According to the invention, the proton pump inhibitors are employed for the treatment of non-cardiac chest pain in the form of ready-to-use medicaments. These medicaments are prepared by methods known per se and familiar to the person skilled In the art. As medicaments, the proton pump inhibitors are either used here as such, or preferably in combination with suitable pharmaceutical excipients or vehicles In the form of tablets, coated tablets, capsules, suppositories, patches (e.g. as TTS), emulsions, suspensions or solutions, the active compound content advantageously being between 0.1 and 95% and it being possible by means of the appropriate choice of the excipients and vehicles to achieve a pharmaceutical administration form adapted exactly to the active compound and/or to the desired onset of action and/or to the duration of action (e.g. a sustained release form or an enteric form).

The person skilled in the art is familiar on the basis of his/her expert knowledge with which excipients or vehicles are suitable for the desired pharmaceutical formulations. Besides solvents, gel-forming agents, suppository bases, tablet excipients and other active compound carriers, it is possible to use, for example, antioxidants, dispersants, emulsifiers, antifoams, taste corrigents, preservatives, solubilizers, colorants or, in particular, permeation promoters and complexing agents (e.g. cyclodextrins).

The active compounds can be administered orally, parenterally or percutaneously.

In general, it has proved advantageous in human medicine to administer the proton pump inhibitor in a daily dose of, in particular, 0.1 to 1.5 mg/kg of body weight, if appropriate in the form of a number of, preferably 1 to 2, individual doses to achieve the desired result. In the case of a parenteral treatment, similar or (in particular in the case of the intravenous administration of the active compounds) as a rule lower dosages can be used. The determination of the optimal dosage and manner of administration of the active compounds necessary in each case can be carried out easily by any person skilled in the art on the basis of his/her expert knowledge.

The invention further relates to a pharmaceutical preparation for the treatment of non-cardiac chest pain, which in an individual dose (tablet, capsule, etc.) contains a proton pump inhibitor as active compound in a dose of between 5 and 100, advantageously between 10 and 60, in particular between 20 and 40 mg.

If the proton pump inhibitors are to be employed for the treatment of non-cardiac chest pain, the pharmaceutical preparations can also contain one or more pharmacologically active constituents of other pharmaceutical groups. Examples which may be mentioned are: tranquillizers (for example from the group consisting of the benzodiazepines, e.g. diazepam), spasmolytics (e.g. bietamiverine or camylofine), anticholinergics (e.g. oxyphencyclimine or phencarbamide), local anaesthetics (e.g. tetracaine or procaine), and optionally also enzymes, vitamins or amino acids.

1. A method of treating non-cardiac chest pain in a patient comprising administering to a patient in need thereof a therapeutically effective amount of a proton pump inhibitor, or a pharmaceutically acceptable salt, hydrate, solvate, hydrate of a salt or solvate of a salt thereof.

2. (canceled)

3. (canceled)

4. (canceled)

5. (canceled)

6. Ready-to-use medicament comprising a proton pump inhibitor as active compound and a reference to the fact that it can be employed for the treatment of non-cardiac chest pain.

7. The method of claim 1 wherein the proton pump inhibitor is selected from the group consisting of 5-methoxy-2-[(4-methoxy-3,5-dimethyl-2-pyridinyl)methylsulphinyl]-1H-benzimidazole (INN: omeprazole), 5-methoxy-2-[(S)-[(4-methoxy-3,5-dimethyl-2-pyridinyl)methyl]sulphinyl]-1H-benzimidazole (INN: esomeprazole), 2-[3-methyl-4-(2,2,2-trifluoroethoxy)-2-pyridinyl)methylsulphinyl]-1H-benzimidazole (INN: lansoprazole), 2-{[4-(3-methoxypropoxy)-2-methylpyridin-2-yl]-methylsulphinyl}-1H-benzimidazole (INN: rabeprazole), 5-difluoromethoxy-2-[(3,4-dimethoxy-2-pyridinyl)methylsulphinyl]-1H-benzimidazole (INN: pantoprazole), (−)-5-difluoromethoxy-2-[(3,4-dimethoxy-2-pyridinyl)methylsulphinyl]-1H-benzimidazole (INN: (−)-pantoprazole) and a pharmaceutically acceptable salt, hydrate, solvate, hydrate of a salt or solvate of a salt thereof.

8. The method of claim 1, wherein the proton pump inhibitor is pantoprazole or a pharmaceutically acceptable salt, hydrate, solvate, hydrate of a salt or solvate of a salt thereof.

9. The method of claim 1, wherein the proton pump inhibitor is selected from the group consisting of pantoprazole sodium sesquihydrate, (−)-pantoprazole sodium sesquihydrate, pantoprazole magnesium dihydrate, omeprazole-magnesium, omeprazole-magnesium tetrahydrate, esomeprazole-magnesium and esomeprazole-magnesium tetrahydrate.
